(19) United States
(12) Patent Application Publication
(10) Pub. No.: US 2023/0165982 A1
(43) Pub. Date: Jun. 1, 2023

(54) VISUALIZATION OF HER2 EXPRESSION IN HUMAN PATIENTS

(71) Applicant: Hober Biotech AB, Stockholm (SE)

(72) Inventors: Sophia Hober, Stockholm (SE); Javad Garousi, Uppsala (SE); Vladimir Tolmachev, Uppsala (SE); Bragina Olga, Tomsk, Tomskaya obl. (RU); Sarah Lindbo, Lidingö (SE); Vladimir Chernov, Tomsk, Tomskaya obl. (RU)

(21) Appl. No.: 17/997,272

(22) PCT Filed: Apr. 30, 2020
(86) PCT No.: PCT/EP2020/061735

§ 371 Date: Oct. 27, 2022

(51) Int. Cl. A61K 51/08

(52) U.S. Cl. CPC A61K 51/088

(57) ABSTRACT

There is provided an imaging agent for use in a method of visualization of HER2 expression in a human patient, said method comprising administering the imaging agent to the patient in a dose of 400-700 μg and subsequently a scanning of the patient to visualize HER2 expression, wherein the imaging agent is a conjugate comprising a radionuclide and a HER2-binding protein (HBP) of a certain amino acid sequence. Further there is provided a unit dose comprising the imaging agent in an amount of 400-700 μg.

### TECHNICAL FIELD

The present disclosure relates to the field of visualization of HER2 expression in human patients.

### BACKGROUND

Human epidermal growth factor receptor 2 (HER2) functions as a molecular target for several therapeutics efficient in the treatment of breast and gastroesophageal cancers. The response to such therapeutics depends on the HER2 expression level, and accurate assessment of HER2 status in tumors is therefore required to avoid under- and overtreatments (Wolff 2013; Bartley 2017). The current standard of care includes the collection of biopsy material followed by an assessment of HER2 status using immunohistochemistry (IHC) and in situ hybridization (ISH) analysis. Tumors with 3+IHC score or 2+IHC and ISH positive are considered as HER2-positive and eligible for HER2-targeting treatment. A major issue of this method is the HER2-expression heterogeneity, and breast cancer patients often have both HER2-positive and HER2-negative metastases (Sörensen 2016; Gebhart 2016). In addition, the invasiveness of biopsies prevents sampling of all metastases, which is associated with a risk of non-representative findings.

Radionuclide molecular imaging of HER2 expression could serve as a non-invasive alternative for patient stratification, offering advantages such as repetitive mapping of HER2 expression in multiple metastases (Tolmachev 2008; Gebhart 2016 review; Mankoff 2016). One promising approach used for the detection of HER2 expression is immunoPET. This strategy utilizes specific recognition of HER2 by monoclonal antibodies as well as superior spatial resolution, registration efficiency and quantification accuracy of positron emission tomography (PET). Therapeutic anti-HER2 antibodies trastuzumab (Dijkers 2010; Laforest 2016; Gebhart 2016; Bensch 2018, Ulaner 2017; Mortimer 2014) and pertuzumab (Ulaner 2018) have both been labeled with the long-lived positron emitters ⁸⁹Zr or ⁶⁴Cu and evaluated in the clinic. Several clinical studies have demonstrated the potential for radionuclide molecular imaging of HER2. For example, ⁸⁹Zr-trastuzumab PET imaging resulted in altered therapeutic decisions for 40% of the patients in cases when clinically relevant lesions could not be biopsied (Bensch 2018). However, the use of full-length antibodies is complicated due to their slow penetration into tumors and slow clearance from the blood. These traits entail the demand of a prolonged delay period between injection and imaging, with the best results obtained 4-8 days after injection (Dijkers 2010; Ulaner 2018). Moreover, the bulky antibodies tend to accumulate in tumors in an unspecific manner, thus creating a risk of false-positive diagnostics.

### SUMMARY

The present inventors have realized that the use of much smaller targeting vectors, such as Engineered Scaffold Proteins (ESPs), is a promising alternative to immunoPET.

ADAPTs are affinity proteins, based on the three-helical scaffold of the albumin-binding domain of streptococcal protein G (Nilvebrant 2013). The small size of ADAPTs and affinities in the low nanomolar range creates promising preconditions for their successful use as imaging agents. A series of ADAPTs has previously been selected for their potential use as HER2-imaging probes (Nilvebrant 2014). To facilitate a rapid clearance of the unbound agent from blood, one particular ADAPT variant, ADAPT6, was developed by eradicating its inherent binding to serum albumin (Nilvebrant 2014).

The objective of the present disclosure is to provide for safe, efficient and accurate visualization of HER2 expression in human patients. After such visualization, the patient can be stratified for HER2-targeting therapies.

Accordingly, there is provided an imaging agent for use in a method of visualization of HER2 expression in a human patient, said method comprising an administration of the imaging agent to the patient in a dose of 400-700 μg and subsequently a scanning of the patient to detect, visualize and/or quantify HER2 expression. Similarly, there is provided a method of visualization of HER2 expression in a human patient, said method comprising an administration of an imaging agent to the patient in a dose of 400-700 μg and subsequently a scanning of the patient to visualize HER2 expression,

There is also provided a unit dose comprising an imaging agent in an amount of 400-700 μg

The imaging agent referred to above is a conjugate comprising a radionuclide and a HER2-binding protein (HBP), wherein the HBP comprises or consists of an amino acid sequence selected from

i) LAX₃AKX₆TX₈X₉Y HLX₁₃X₁₄X₁₅GVX₁₈DX₂₀ YKX₂₃LIDKX₂₈KT VEX₃₃VX₃₅AX₃₇YX₃₉X₄₀ ILX₄₃ALP (SEQ ID NO:18), wherein, independently of each other,
X₃ is selected from A, G, P, S and V;
X₆ is selected from D and E;
X₈ is selected from A and V;
X₉ is selected from L and N;
X₁₃ is selected from D and T;
X₁₄ is selected from K and R;
X₁₅ is selected from I, L, M, T and V;
X₁₈ is selected from S and A;
X₂₀ is selected from F, Y and A;
X₂₃ is selected from D and R;
X₂₈ is selected from A and V;
X₃₃ is selected from G, S and D;
X₃₅ is selected from K, M and R;
X₃₇ is selected from L and R;
X₃₉ is selected from A, F and L;
X₄₀ is selected from A and E; and
X₄₃ is selected from A, H, K, P, R, T, Q and Y;
   and ii) an amino acid sequence which has at least 95% identity to the sequence defined in i).

In an embodiment, the radionuclide is coupled to a terminal end of the HBP, such as the N-terminal end of the HBP. The imaging agent may further comprise a linking amino acid sequence, wherein the radionuclide is coupled to the terminal end of the HBP via the linking amino acid sequence.

In an embodiment, the number of amino acid residues of the linking amino acid sequence is 5-30, such as 5-20.

In an embodiment, at least part of the linking amino acid sequence forms a chelator for the radionuclide. The chelator may comprise the sequence HHHHHH (SEQ ID NO:3).

In an embodiment, the linking amino acid sequence distances any chelator or other radionuclide-binding moiety from the HBP by at least five amino acid residues, such as at least six amino acid residues.

In an embodiment of amino acid sequence i):
X₃ is selected from A, G, P;
X₆ is E;
X₉ is L;
X₁₃ 1 S D;
X₁₄ is R;
X₁₅ is selected from L and V;
X₁₈ is selected from S and A;
X₂₀ is selected from F, Y and A;
X₂₈ is A;
X₃₃ is G;
X₃₅ is selected from K and R;
X₃₇ is L;
X₃₉ is selected from F and L;
X₄₀ is E; and
X₄₃ is selected from H, P and R.

In an embodiment, the HBP comprises or consists of an amino acid sequence selected from the group consisting of:

(SEQ ID NO: 6)
LAAAKETALY HLDRLGVADA YKDLIDKAKT VEGVKARYFE ILHALP;
(SEQ ID NO: 7)
LAAAKETALY HLDRVGVSDY YKDLIDKAKT VEGVRALYLE ILPALP;
(SEQ ID NO: 8)
LAPAKETALY HLDRVGVSDY YKDLIDKAK TVEGVRALYFE ILHALP;
(SEQ ID NO: 9)
LAAAKETALY HLDRLGVSDY YKDLIDKAK TVEGVKALYFE ILHALP;
(SEQ ID NO: 10)
LAPAKETALY HLDRLGVSDY YKDLIDKAK TVEGVRALYLE ILKALP;
(SEQ ID NO: 11)
LAGAKETALY HLDRLGVSDY YKDLIDKAK TVEGVRALYLE ILTALP;
(SEQ ID NO: 12)
LAPAKETALY HLDRLGVSDY YKDLIDKAK TVEGVRALYFE ILRALP;
(SEQ ID NO: 13)
LAGAKETALY HLDRVGVSDY YKDLIDKAK TVEGVRALYLE ILRALP;
(SEQ ID NO: 14)
LAAAKETALY HLDRVGVSDY YKDLIDKAK TVEGVMALYAE ILPALP;
(SEQ ID NO: 15)
LAGAKETALY HLDKTGVSDY YKDLIDKAK TVEGVRALYLE ILQALP;
(SEQ ID NO: 16)
LAAAKETALY HLTRVGVSDY YKDLIDKAK TVEGVRALYFE ILYALP;
and
(SEQ ID NO: 17)
LASAKDTALY HLDRVGVSDY YKDLIDKAK TVEGVRALYAE ILAALP.

In an embodiment, the HBP comprises or consist of an amino acid sequence selected from the group consisting of:

(SEQ ID NO: 6)
LAAAKETALY HLDRLGVADA YKDLIDKAKT VEGVKARYFE ILHALP;
(SEQ ID NO: 9)
LAAAKETALY HLDRLGVSDY YKDLIDKAK TVEGVKALYFE ILHALP;
and
(SEQ ID NO: 13)
LAGAKETALY HLDRVGVSDY YKDLIDKAK TVEGVRALYLE ILRALP.

In an embodiment, the radionuclide is selected from the group consisting of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y, ^{110m}In, ¹²³I, ¹³¹I, ^{99m}Tc, ¹¹¹In and ⁶⁷Ga.

In an embodiment, the radionuclide is selected from the group consisting of ¹⁸F, ⁶⁸Ga, ^{99m}Tc and ¹¹¹In.

In an embodiment, the radionuclide is selected from the group consisting of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y and ^{110m}In and the scanning is PET.

In an embodiment, the radionuclide is ¹⁸F or ⁶⁸Ga and the scanning is PET.

In an embodiment, the radionuclide is conjugated to the HBP by means of a chelator or a prosthetic group forming a covalent bond to the radionuclide.

In an embodiment, the imaging agent comprises less than 73 amino acid residues, such as less than 68 amino acid residues.

In an embodiment, the imaging agent is administered by intravenously.

In an embodiment, the above-mentioned scanning is carried out within 4 hours of the administration of the imaging agent, such as within 3 hours of the administration of the imaging agent.

In an embodiment, the above-mentioned scanning is carried out between 1 and 3 hours after the administration of the imaging agent, such as between 1.5 and 2.5 hours after the administration of the imaging agent.

In an embodiment, the radionuclide is selected from the group consisting of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y and ^{110m}In and the scanning is PET carried out between 1 and 3 hours after the administration of the imaging agent, such as between 1.5 and 2.5 hours after the administration of the imaging agent.

In an embodiment, the patient suffers from a breast cancer or a gastroesophageal cancer.

In an embodiment, the above-mentioned dose is 400-600 μg, such as 450-550 μg, such as about 500 μg. Similarly, the amount the imaging agent in the unit dose may be 400-600 μg, such as 450-550 μg, such as about 500 μg.

In an embodiment, the imaging agent is formulated in a composition adapted for intravenous administration. The volume of the composition may be 1-15 ml, such as 1-10 ml, such as 8-10 ml. The composition may be water-based, such as saline-based. The water-based composition may be buffered, such as phosphate-buffered.

There is also provided a product comprising a container and the above-mentioned unit dose, wherein the unit dose is contained in the container. The container may be a vial or ampoule. The volume of the container may be 1-15 ml, such as 1-10 ml, such as 8-10 ml.

### BRIEF DESCRIPTION OF THE DRAWINGS

FIG. 1 shows whole-body images at 2, 4, 6 and 24 h after injection of 500 μg ^{99m}Tc-ADAPT6 in patient 1 in Examples section below.

FIG. 2 shows the kinetics of elimination of ^{99m}Tc-ADAPT6 from blood.

FIG. 3 shows primary tumor-to-contralateral site ratio at 2 h after injection of 250 μg ^{99m}Tc-ADAPT6. FIG. 3 further shows primary tumor-to-contralateral site ratio at 2, 4 and 6 h after injection of 500 and 1000 μg ^{99m}Tc-ADAPT6.

FIG. 4 shows representative anterior images of patients with HER2 negative and HER2 positive tumors after injection of 250, 500 or 1000 μg ^{99m}Tc-ADAPT6.

FIG. 5 shows tumor sites visualization with planar scintigraphy in patient 4: (A) ^{99m}Tc-ADAPT6; (B) ^{99m}Tc-pyrophosphate at the time of imaging with ^{99m}Tc-ADAPT6; (C) ^{99m}Tc-pyrophosphate 6 months after ADAPT6 injection.

FIG. 6 shows tumor-to-liver ratio at 2, 4 and 6 h after injection of 500 and 1000 μg ^{99m}Tc-ADAPT6.

### DETAILED DESCRIPTION

As a first aspect of the present disclosure, there is provided an imaging agent for use in a method of visualization of HER2 expression in a human patient, which patient typically suffers from a breast cancer or a gastroesophageal cancer. It may also be a patient with suspected recurrent breast or gastroesophageal cancer.

The method comprises an administration of the imaging agent to the patient in a dose of 400-700 μg. Preferably, the dose is 400-600 μg, such as 450-550 μg, such as about 500 μg. The route of administration is typically intravenous.

Subsequent to the administration of the imaging agent, the patient is scanned to detect, visualize and/or quantify HER2 expression. The imaging agent of the present disclosure provides for high-contrast imaging relatively quickly, which reduces the time the patient has to stay in the clinic (which in turn reduces costs and improve the patient's quality of life). Hence, the patient is patient is preferably scanned within 4 hours of the administration of the imaging agent, such as within 3 hours of the administration of the imaging agent. In an embodiment, the scanning is carried out between 1 and 3 hours after the administration of the imaging agent, such as between 1.5 and 2.5 hours after the administration of the imaging agent. The scanning is typically a tomography, preferably positron emission tomography (PET) or single-photon emission computed tomography (SPECT). For the latter, a CZT-based camera technology may be used.

The imaging agent is a conjugate comprising a radionuclide and a HER2-binding protein (HBP).

In an embodiment, the radionuclide is selected from the group consisting of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y, ^{110m}In, ¹²³I, ¹³¹I, ^{99m}Tc, ¹¹¹In and ⁶⁷Ga. A preferred group consists of ¹⁸F, ⁶⁸Ga, ^{99m}Tc and ¹¹¹In. Another preferred group consists of ¹⁸F, ⁶⁸Ga and ¹¹¹In.

For radiolabelling with ¹⁸F, a prosthetic group (forming a covalent bond to ¹⁸F) may be coupled to the HBP (optionally via the linking amino acid sequence discussed below). Examples of resulting structures are N-(2-(4-[¹⁸F]-fluorobenzamido)ethyl)maleimido ([¹⁸F]FBEM), 4-[¹⁸F]-fluorobenzaldehyde ([¹⁸F]-FBA) and [¹⁸F]-fluorophenyloxadiazole methylsulfone ([¹⁸F]-FPOS. Another option is [¹⁸F]aluminium monofluoride in combination with a triaza chelator.

Also in case of radiolabeling with ¹²³I, ¹²⁴I, ¹³¹I and ⁷⁶Br, a prosthetic group may be used. Examples of resulting structures are iodo-/bromo-benzoate and iodo-/bromo-hydroxyphenylethyl maleimide.

For radiolabeling with ⁶⁸Ga, ⁶⁷Ga, ⁶⁶Ga, ⁴⁴Sc, ⁵⁵Co, ⁴¹Ti, ⁸⁶Y, ^{110m}In and ¹¹¹In, it is preferred to couple a chelator to the HBP (optionally via the linking amino acid sequence discussed below). Examples of chelators are DOTA, NOTA, NODAGA and DOTAGA and their derivatives.

For ⁶¹Cu and ⁶⁴Cu, a cross-bridged chelator, such as CB-TE2A, is a better option.

For radiolabelling with ^{99m}Tc, a variety of chelators can be used, such as hexahistidine (H₆) and chelators based on a cysteine- or mercaptoacetyl-containing peptide.

In case of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y or ^{110m}In, the scanning technique is preferably PET.

In case of ¹²³I, ¹³¹I, ^{99m}Tc, ¹¹¹In or ⁶⁷Ga, the scanning technique preferably comprises SPECT, e.g. using a CZT-based camera.

The radionuclide is preferably coupled to a terminal end of the HBP, such as the N-terminal end of the HBP. In an embodiment, the imaging agent further comprises a linking amino acid sequence and the radionuclide is coupled to the terminal end of the HBP via the linking amino acid sequence. The number of amino acid residues of the linking amino acid sequence is typically 5-30, preferably 5-25 or 5-20.

In an embodiment, at least part of the linking amino acid sequence forms a chelator for the radionuclide. As an example, the chelator-forming part may comprise the sequence HHHHHH (SEQ ID NO:3), which can bind ^{99m}Tc. An alternative to HHHHHH is HEHEHE (SEQ ID NO:5).

The linking amino acid sequence preferably distances any chelator or other radionuclide-binding moiety from the HBP, e.g. by at least five amino acid residues, such as at least six amino acid residues. Thereby, any interference with the HER2-binding may be avoided or at least reduced. In an embodiment, the linking amino acid sequence comprises the sequence DEAVDANS (SEQ ID NO:4) on the C-terminal side of the chelator or radionuclide-binding moiety for such distancing. Accordingly, linking amino acid sequence may comprise both SEQ ID NO:3 and SEQ ID NO:4, e.g. forming SEQ ID NO:2.

The HBP comprises or consists of an amino acid sequence selected from i) LAX₃AKX₆TX₈X₉Y HLX₁₃X₁₄X₁₅GVX₁₈DX₂₀ YKX₂₃LIDKX₂₈KT VEX₃₃VX₃₅AX₃₇YX₃₉X₄₀ ILX₄₃ALP, wherein, independently of each other,
X₃ is selected from A, G, P, S and V, preferably A, G and P;
X₆ is selected from D and E, preferably E;
X₈ is selected from A and V;
X₉ is selected from L and N, preferably L;
X₁₃ is selected from D and T, preferably D;
X₁₄ is selected from K and R, preferably R;
X₁₅ is selected from I, L, M, T and V, preferably L and V;
X₁₈ is selected from S and A;
X₂₀ is selected from F, Y and A;
X₂₃ is selected from D and R;
X₂₈ is selected from A and V, preferably A;
X₃₃ is selected from G, S and D, preferably G;
X₃₅ is selected from K, M and R, preferably K and R;
X₃₇ is selected from L and R, preferably L;
X₃₉ is selected from A, F and L, preferably F and L;
X₄₀ is selected from A and E, preferably E; and
X₄₃ is selected from A, H, K, P, R, T, Q and Y, preferably H, P and R; and ii) an amino acid sequence which has at least 95% identity to the sequence defined in i).

Data supporting binding activity of i) and ii) to HER2 is presented in WO2014076179, Nilvebrant 2014 and the Examples section below.

In a preferred embodiment of the amino acid sequence i)
X₃ is selected from A, G, P, preferably A and G;
X₆ is E;
X₈ is A and V;
X₉ is L;
X₁₃ is D;
X₁₄ is R;
X₁₅ is selected from L and V;
X₁₈ is selected from S and A;
X₂₀ is selected from F, Y and A;
X₂₃ is selected from D and R;
X₂₈ is A;
X₃₃ is G;
X₃₅ is selected from K and R;
X₃₇ is L;
X₃₉ is selected from F and L;
X₄₀ is E; and
X₄₃ is selected from H, P and R.

In another preferred embodiment, the HBP comprises or consists of an amino acid sequence selected from the group consisting of:

(SEQ ID NO: 6)
LAAAKETALY HLDRLGVADA YKDLIDKAKT VEGVKARYFE ILHALP;
(SEQ ID NO: 7)
LAAAKETALY HLDRVGVSDY YKDLIDKAKT VEGVRALYLE ILPALP;
(SEQ ID NO: 8)
LAPAKETALY HLDRVGVSDY YKDLIDKAKT VEGVRALYFE ILHALP;
(SEQ ID NO: 9)
LAAAKETALY HLDRLGVSDY YKDLIDKAK TVEGVKALYFE ILHALP;
(SEQ ID NO: 10)
LAPAKETALY HLDRLGVSDY YKDLIDKAK TVEGVRALYLE ILKALP;
(SEQ ID NO: 11)
LAGAKETALY HLDRLGVSDY YKDLIDKAK TVEGVRALYLE ILTALP;
(SEQ ID NO: 12)
LAPAKETALY HLDRLGVSDY YKDLIDKAK TVEGVRALYFE ILRALP;
(SEQ ID NO: 13)
LAGAKETALY HLDRVGVSDY YKDLIDKAK TVEGVRALYLE ILRALP;
(SEQ ID NO: 14)
LAAAKETALY HLDRVGVSDY YKDLIDKAK TVEGVMALYAE ILPALP;
(SEQ ID NO: 15)
LAGAKETALY HLDKTGVSDY YKDLIDKAK TVEGVRALYLE ILQALP;
(SEQ ID NO: 16)
LAAAKETALY HLTRVGVSDY YKDLIDKAK TVEGVRALYFE ILYALP;
and
(SEQ ID NO: 17)
LASAKDTALY HLDRVGVSDY YKDLIDKAK TVEGVRALYAE ILAALP.

A particularly preferred group consists of SEQ ID NO:6, SEQ ID NO:9 and SEQ ID NO:13. SEQ ID NO:9 and 13 were identified by both phage display and FACS in Nilvebrant 2014, which the inventors consider to be beneficial. SEQ ID NO:6 is used in the Examples section below. The HBP and the above-mentioned linking amino acid sequence may be fused to consist of SEQ ID NO:1.

In an embodiment, the imaging agent comprises less than 73 amino acid residues, such as less than 68 amino acid residues. Such a relatively small size facilitates high tumor uptake (further discussed below) and thus high-contrast imaging. The total molecular weight of the therapeutic conjugate is preferably below 12.0 kDa, preferably below 8.0 kDa, such as below 7.1 kDa.

In an embodiment, the HER2 expression in the patient is quantified subsequent to the scanning and if the quantified HER2 expression is found to be above a clinically relevant reference value, a HER2-targeting treatment is applied. If the quantified HER2 expression is below the reference value, the decision may be to refrain from the HER2-targeting treatment.

As a second aspect of the present disclosure, there is provided a unit dose comprising the imaging agent of the first aspect in an amount of 400-700 μg. Preferably, the amount is 400-600 μg, such as 450-550 μg, such as about 500 μg. The embodiments of the first aspect apply to the second aspect mutatis mutandis. The unit dose of the second aspect facilitates the method of the first aspect.

The imaging agent of the second aspect is preferably formulated in a composition adapted for intravenous administration.

The composition is typically water-based, such as saline-based. The water-based composition may be buffered, such as phosphate-buffered. Accordingly, the composition may comprise phosphate-buffered saline (PBS). As an example, a PBS-based buffer is a suitable buffer when the radionuclide is ^{99m}Tc. As another example, the pH of the composition is preferably about 5 when the radionuclide is ¹¹¹In.

The unit dose of the second aspect may be ready for administration, preferably intravenous administration. Alternatively, the unit dose may be subjected to purification prior to administration. Such a purification is typically carried out in or in close connection to the clinic.

Whether or not the purification is required may depend to the nature of the radiolabel. Radiohalogens usually require the purification. A labelling using radiometals might be optimized to such extent that purification of a product is not required. Examples of radiometals for which purification is generally not required are ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y, ^{110m}In, ^{99m}Tc, ¹¹¹In and ⁶⁷Ga.

The purification may comprise the steps of: loading of a solution of the imaging agent on a disposable sterilizable size-exclusion column (cartridge) followed by elution with an appropriate solvent, for example PBS. The column (cartridge) should be pre-calibrated to determine the dead volume and the volume of eluent necessary for elution of the high-molecular weight fraction without the low-molecular weight fraction. The eluate containing the high-molecular-weight fraction is collected.

The volume to be administrated is typically 1-15 ml, such as 1-10 ml, such as 8-10 ml. Accordingly, the volume of the composition may be 1-15 ml, such as 1-10 ml, such as 8-10 ml, in particular when no purification is required.

The embodiments of the second aspect apply to the first aspect mutatis mutandis.

As a third aspect of the present disclosure, there is provided a product comprising a container and the unit dose of the second aspect, wherein the unit dose is contained in the container. Such a product, which is typically a single-use product (one product per patient and visualization), facilitates the procedures in the clinic. The container is typically a vial or ampoule. The volume of the container may be 1-15 ml, such as 1-10 ml, such as 8-10 ml.

As a fourth aspect of the present disclosure, there is provided a method of visualization of HER2 expression in a human patient, said method comprising an administration of an imaging agent to the patient in a dose of 400-700 μg and subsequently a scanning of the patient to visualize HER2 expression. The imaging agent is the same as in the first aspect. Embodiments of the fourth aspect are derived from the above description of the first aspect.

### Examples

In an in-human study, an imaging agent (referred to as “^{99m}Tc-ADAPT6” below) has been evaluated in patients with primary HER2-positive and HER2-negative breast cancer.

The primary objectives of the study were:
a. To assess the distribution of ^{99m}Tc-ADAPT6 in normal tissues and tumors over time;
b. To evaluate the dosimetry of ^{99m}Tc-ADAPT6;
c. To obtain initial information concerning safety and tolerability of ^{99m}Tc-ADAPT6 after single intravenous injection:

A secondary objective was to compare the tumor imaging data with the data concerning HER2 expression obtained by immunohistochemistry (IHC) or fluorescent in situ hybridization (FISH) analysis of biopsy samples.

In the study, human patients were injected with 250, 500 or 1000 μg of ^{99m}Tc-ADAPT6. Evaluations through planar scintigraphy and PET imaging were carried out 2, 4, 6 and 24 h after injection. However, the patients injected with 250 μg were only evaluated after 2 h.

### Materials and Methods

Patients

This was a prospective, open-label, non-randomized Phase I diagnostic study in patients with untreated primary breast cancer. The protocol was approved by the Scientific Council of Cancer Research Institute, Tomsk National Research Medical Center of the Russian Academy of Sciences. All patients signed a written informed consent. Twenty-eight (28) patients were enrolled (Table 1).

TABLE 1
Patient Characteristics Before Injection with
^{99m}Tc-ADAPT6.

| | | HER2 status | Primary | |
|---|---|---|---|---|
| Patient | Age | of primary | tumor | |
| no | (y) | tumor | ER/PgR | Stage |
| | 500 μg; mean tumor size 23 ± 8 mm | | | |
| 1 | 36 | 3+ (ICH) | ER+/PgR − | IIB (T₂N₁M₀) |
| 2 | 63 | 3+ (ICH) | ER−/PgR− | IIIA (T₃N₁M₀) |
| 3 | 50 | 3+ (ICH) | ER+/PgR− | IIIA (T₂N₂M₀) |
| 4 | 61 | 3+ (ICH) | ER−/PgR− | IIB (T₂N₁M₀) |
| 5 | 64 | 2+ (ICH)/ | ER−/PgR− | IIIB (T₂N₁M₀)/ |
| | | FISH+ | | IV (T₂N₃M₁) * |
| 6 | 34 | 0 (ICH) | ER+/PgR+ | I (T₁N₀M₀) |
| 7 | 47 | 0 (ICH) | ER+; /PgR+ | IIA (T₂N₀M₀) |
| 8 | 41 | 0 (ICH) | ER+/PgR+ | IIA (T₂N₀M₀) |
| 9 | 63 | 1+ (ICH) | ER+/PgR+ | IIB (T₂N₁M₀) |
| 10 | 59 | 1+ (ICH) | ER+/PgR− | IIA (T₂N₀M₀) |
| 11 | 40 | 0 (ICH) | ER−/PgR− | IIIA (T₃N₁M₀) |
| | 1000 μg; mean tumor size 31 ± 11 mm | | | |
| 12 | 34 | 3+ (ICH) | ER+/PgR− | IIA (T₂N₀M₀) |
| 13 | 37 | 3+ (ICH) | ER+/PgR− | IIA (T₁N₁M₀) |
| 14 | 43 | 2+ (ICH)/ | ER+/PgR+ | IIA (T₂N₀M₀) |
| | | FISH+ | | |
| 15 | 36 | 3+ (ICH) | ER+/PgR+ | IIA (T₁N₁M₀) |
| 16 | 33 | 3+ (ICH) | ER−/PgR− | IIA (T₂N₀M₀) |
| 17 | 58 | 3+ (ICH)/ | ER+/PgR+ | I (T₁N₀M₀) |
| | | FISH− ** | | |
| 18 | 51 | 1+ (ICH) | ER+/PgR− | IIA (T₂N₀M₀) |
| 19 | 63 | 1+ (ICH) | ER+/PgR+ | IIA (T₂N₀M₀) |
| 20 | 62 | 0 (ICH) | ER−/PgR− | IIA (T₂N₀M₀) |
| 21 | 71 | 1+ (ICH) | ER+/PgR− | IIA (T₂N₀M₀) |
| 22 | 42 | 0 (ICH) | ER+/PgR+ | IIA (T₂N₀M₀) |
| | 250 μg; mean tumor size 30 ± 10 mm | | | |
| 23 | 51 | 2+ (ICH)/ | ER+/PgR-; | IIA (T₂N₀M₀) |
| | | FISH+ | | |
| 24 | 48 | 2+ (ICH)/ | ER+/PgR− | IIA (T₂N₀M₀) |
| | | FISH+ | | |
| 25 | 61 | 2+ (ICH)/ | ER+/PgR+ | IIA (T4N₁M₀) |
| | | FISH+ | | |
| 26 | 39 | 3+ (ICH) | ER+/PgR− | IIA (T₂N₀M₀) |
| 27 | 29 | 2+ (ICH)/ | ER+/PgR+ | IIA (T₂N₀M₀) |
| | | FISH− | | |
| 28 | 62 | 1+(IHC) | ER+/PgR+ | I (T₁N₀M₀) |

* staging has been changed as imaging revealed distant metastases;
** FISH analysis after imaging confirmed HER2-negative status.

Biopsy samples of primary tumors were collected, and the level of HER2 expression was determined by immunohistochemistry (IHC) using Herceptest (DAKO). For the tumors with a score of 2+ or in a case of questionable results, a HER2 amplification was assessed using fluorescent in situ hybridization (FISH). The tumors were classified as HER2-positive (HercepTest score 3+ or HercepTest score 2+ and FISH-positive) or HER2-negative (HercepTest score 0 or 1+, or score 2+ but FISH-negative) according to the guidelines of the American Society of Clinical Oncology (Wolff 2013).

As a local standard of care, a mammography (Giotto Image), a bone scan (Siemens E.Cam 180) using ^{99m}Tc-pyrophosphate, a chest CT (Siemens Somatom Emotions 16 ECO) and an ultrasound (GE LOGIQ E9) imaging were performed for all patients. For patient 4, an additional MRI (Siemens Magnetom Essenza 1.5T) examination was performed.

Imaging Protocol

Labeling of ADAPT6 was performed in aseptic conditions according to a method described earlier (Lindbo 2016). Briefly, ^{99m}Tc was converted to ^{99m}Tc(H₂O)₃(CO)₃+ using a CRS (Center for Radiopharmaceutical Sciences) kit. PBS (100 μL) and ^{99m}Tc(H₂O)₃(CO)₃+ (400 μL, 1.3±0.3 GBq) were added to vials containing either 250, 500 or 1000 μg freeze-dried protein having the sequence GSSHHHHHHD EAVDANSLAA AKETALYHLD RLGVADAYKD LIDKAKTVEG VKARYFEILH ALP (SEQ ID NO:1), which is ADAPT6 (SEQ ID NO:6) with the N-terminal extension GSSHHHHHHD EAVDANS (SEQ ID NO:2). In the N-terminal extension sequence, the hexahistidine (HHHHHH (SEQ ID NO:3)) subsequence is a chelator for the radionuclide (^{99m}Tc). The DEAVDANS (SEQ ID NO:4) subsequence acts as a spacer between the chelating moiety and the HER2-binding protein. Further, it facilitates production of the protein. The vials were incubated for 60 min at 50° C., and radiolabeled protein (“^{99m}Tc-ADAPT6”) was purified by size-exclusion chromatography. The yield was 77±9%, and radiochemical purity was 99±1%.

^{99m}Tc-ADAPT6 was injected as an intravenous bolus (a high-molecular-weight fraction from size-exclusion purification (solution in PBS) that had been diluted with sterile saline to a volume of 10 ml). Patients 1-11 were injected with 500 μg ADAPT6 (416±135 MBq), and patients 12-22 with 1000 μg (349±133 MBq) Imaging was performed using (Siemens E.Cam 180) scanner. Planar whole-body imaging and SPECT scans were performed at 2, 4, 6 and 24 h. Patients 23-28 were injected with 250 μg (165±29 MBq), and planar whole-body imaging and SPECT scans were performed at 2 h.

Monitoring of vital signs and possible side effects was performed during imaging study (0-24 h after injection) and 3-7 days after injection. Blood and urine analyses were performed 5 and 14 days after injection.

Assessment of Distribution and Dosimetry

Regions of interest (ROI) were drawn over organs of interest and the whole body, on the anterior and posterior whole-body images of patients injected with 500 and 1000 μg ^{99m}Tc-ADAPT6; a geometric mean at 2, 4, 6 and 24 h was calculated for each ROI. For quantification, a counting of known activity of ^{99m}Tc in a water-filled phantom in combination with Chang's correction was used. To assess the kinetics in blood, an ROI was placed over the heart content. The data were fitted by a single exponential function, and residence time was calculated as an area under the fitted curve using Prism 8 for window software (GraphPad Software, LLC). Absorbed doses were calculated by OLINDA/EXM 1.1 using Adult Female phantom.

To calculate the tumor-to-contralateral breast and the tumor-to-liver ratios, a 3.5-cm³ volume of interest (VOI) was drawn on a tomographic image in the area of the highest tumor uptake, and the counts were recorded. Thereafter, this VOI was copied to a contralateral breast and liver to obtain counts in the reference areas.

Statistics

Values are reported as a mean±standard deviation. The significance of the differences between uptake in organs at different time points was analyzed using 1-way ANOVA. The significance of the differences between tumor-to-contralateral breast and tumor-to-liver ratio values for HER2-positive and HER2-negative tumors was analyzed using the nonparametric Mann-Whitney U test. A 2-sided P value of less than 0.05 was considered significant.

### Results

Safety and Tolerability

^{99m}Tc-ADAPT6 was administered in twenty-eight patients. The administration was well tolerated. No drug-related adverse reactions or changes in vital signs were observed during imaging or the follow-up period. No changes in blood or urine analyses were detected.

Distribution and Dosimetry

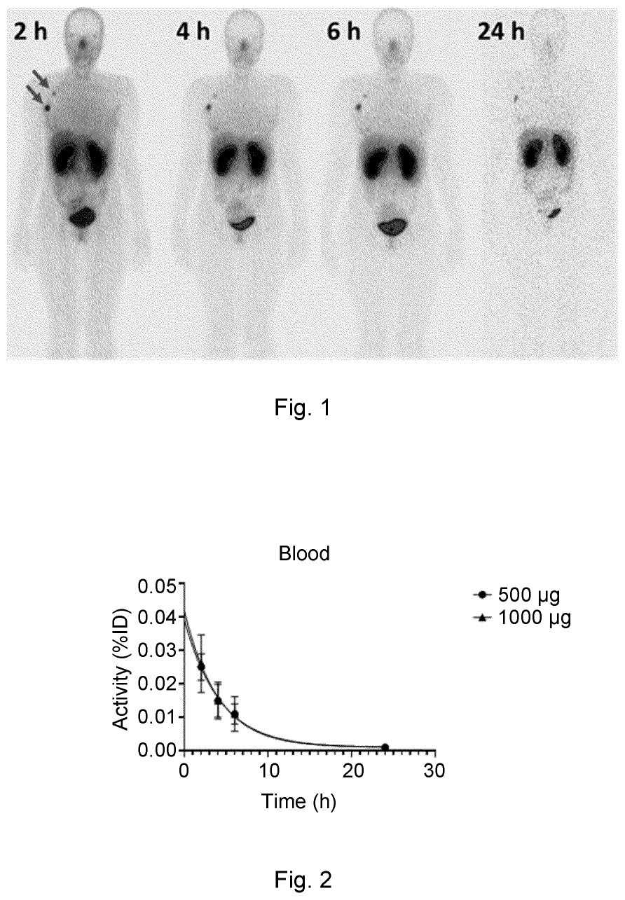

The highest uptake in normal organs was observed in the kidneys, liver and lungs (FIG. 1 and Table 2). Moderate activity was observed in the gastrointestinal tract content. Uptake in salivary and lacrimal glands was also visualized. Distribution of the activity was very similar after injection of 500 μg and 1000 μg. The only significant difference (p<0.05) between the two doses was found when comparing uptake in the intestines content at 6 and 24 h after injection, where the uptake was lower for the 500 μg dose. The decay-corrected uptake in the kidneys, liver, lungs and intestines content reached a plateau at 2 h after injection.

TABLE 2
Uptake of ^{99m}Tc in Tumor-Free Areas of Organs with Highest
Uptake on SPECT Images After Injection ^{99m}Tc-ADAPT6 (decay
corrected). Data are presented as percent injected radioactivity
per organ (mean values and SD from all patients).

| | Kidney | | | Lungs | | |
|---|---|---|---|---|---|---|
| | 250 μg | 500 μg | 1000 μg | 250 μg | 500 μg | 1000 μg |
| 2 h | 20 ± 10 | 27 ± 10 | 35 ± 9 | 2.7 ± 0.9 | 3.3 ± 0.8 | 2.7 ± 0.6 |
| 4 h | | 31 ± 12 | 36 ± 10 | | 2.5 ± 0.8 | 2.2 ± 0.4 |
| 6 h | | 32 ± 9 | 45 ± 11 | | 2.0 ± 0.6^{c} | 2.0 ± 0.4^{c} |
| 24 h | | 29 ± 10 | 38 ± 8 | | 1.4 ± 0.5^{b} | 1.2 ± 0.4^{b} |

| | Liver | | | Small intestines | | |
|---|---|---|---|---|---|---|
| | 250 μg | 500 μg | 1000 μg | 250 μg | 500 μg | 1000 μg |
| 2 h | 3.1 ± 0.3 | 3.2 ± 1.1 | 2.4 ± 0.8 | 1.8 ± 0.4 | 0.8 ± 0.3 | 1.0 ± 0.3 |
| 4 h | | 2.8 ± 1.1 | 2.4 ± 1.0 | | 0.9 ± 0.3 | 1.3 ± 0.5 |
| 6 h | | 2.6 ± 0.8 | 2.0 ± 0.7 | | 0.8 ± 0.3^{a} | 1.3 ± 0.5 |
| 24 h | | 2.4 ± 1.0 | 1.8 ± 0.8 | | 0.6 ± 0.2^{a} | 1.0 ± 0.3 |

^{a}Significantly (p < 0.05) lower uptake in the intestines content after injection of 500 μg compared to 1000 μg;
^{b}Significantly (p < 0.05) lower uptake in the lungs at 24 h after injection compared to 2 and 4 h;
^{c}Significantly (p < 0.05) lower uptake in the lungs at 6 h after injection compared to 2 h.

The blood kinetics of ^{99m}Tc-ADAPT6 is shown in FIG. 2. The elimination rates for 500 μg (half-life 3.1 h, 95% CI from 2.4 to 4.0 h) and 1000 μg (half-life 3.0 h, 95% CI from 2.3 to 3.9 h) were similar.

Estimated absorbed doses are presented in Table 3. The highest absorbing organ was kidney. Absorption in adrenal, gall bladder wall, liver, spleen and pancreas were also noticeable, although they were several-fold lower than the renal dose. Doses to adrenals, stomach wall, spleen, thyroid and uterus were significantly (p<0.05) higher for 1000 μg, but absolute difference was prominent only for adrenal and thyroid. Total effective dose was 0.009±0.002 mSv/MBq for 500 μg and 0.010±0.003 mSv/MBq for 1000 μg. For a typical injected activity in this study, 380 MBq, this would result in an effective dose of 3.4 and 3.8 mSv.

TABLE 3
Absorbed doses after injection of 500 and 1000 μg.

| | 500 μg | 1000 μg |
|---|---|---|
| Adrenals | 0.023 ± 0.005* | 0.032 ± 0.009 |
| Brain | 0.001 ± 0.000 | 0.001 ± 0.000 |
| Breasts | 0.007 ± 0.002 | 0.0o9 ± 0.005 |
| Gallbladder wall | 0.013 ± 0.008 | 0.012 ± 0.003 |
| Lower large intestine | 0.005 ± 0.001 | 0.005 ± 0.001 |
| wall | | |
| Small intestine | 0.006 ± 0.001 | 0.008 ± 0.002 |
| Stomach wall | 0.006 ± 0.001* | 0.008 ± 0.002 |
| Upper large intestine | 0.007 ± 0.001 | 0.008 ± 0.002 |
| wall | | |
| Heart wall | 0.004 ± 0.001 | 0.004 ± 0.001 |
| Kidney | 0.135 ± 0.042 | 0.191 ± 0.047 |
| Liver | 0.011 ± 0.008 | 0.008 ± 0.002 |
| Lungs | 0.005 ± 0.001 | 0.006 ± 0.001 |
| Muscle | 0.003 ± 0.000 | 0.003 ± 0.001 |
| Ovaries | 0.008 ± 0.002 | 0.010 ± 0.003 |
| Pancreas | 0.011 ± 0.002 | 0.014 ± 0.004 |
| Red Marrow | 0.004 ± 0.001 | 0.005 ± 0.001 |
| Osteogenic Cells | 0.006 ± 0.001 | 0.008 ± 0.002 |
| Skin | 0.001 ± 0.000 | 0.002 ± 0.000 |
| Spleen | 0.011 ± 0.003* | 0.015 ± 0.004 |
| Thymus | 0.005 ± 0.002 | 0.006 ± 0.002 |
| Thyroid | 0.009 ± 0.004* | 0.014 ± 0.005 |
| Urinary bladder wall | 0.012 ± 0.007 | 0.012 ± 0.006 |
| Uterus | 0.005 ± 0.001* | 0.007 ± 0.002 |
| Total body | 0.004 ± 0.001 | 0.005 ± 0.001 |
| Effective Dose | 0.017 ± 0.004 | 0.022 ± 0.005 |
| equivalent (mSv/MBq) | | |
| Effective Dose | 0.009 ± 0.002 | 0.010 ± 0.003 |
| (mSv/MBq) | | |

Data presented as mean mGy/MBq ± SD (n = 9).
*Significant (p < 0.05) difference between doses after injection of 500 and 1000 μg

Discrimination Between Tumors with High and Low HER2 Expression

Unexpectedly, all tumors and involved lymph nodes with both high and low HER2 expression were clearly visualized already 2 h after injection of 250, 500 or 1000 μg ^{99m}Tc-ADAPT6, and remained visible throughout the study (FIGS. 1 and 4).

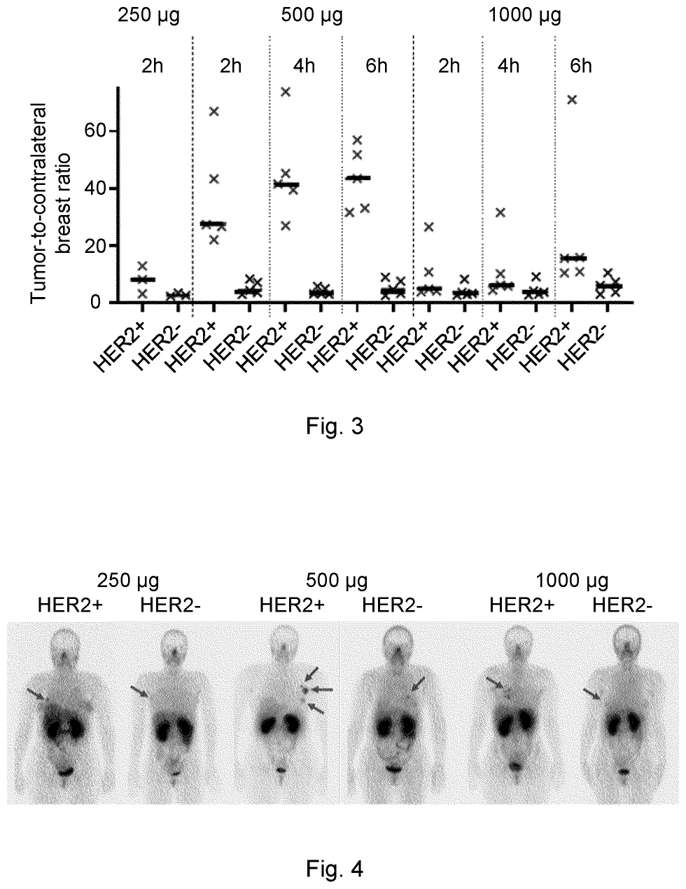

Also unexpectable, the best discrimination between tumors with high and low HER2 expression was provided in the case of injection of 500 μg ^{99m}Tc-ADAPT6. The mean value of tumor-to-contralateral breast ratio value for HER2-positive tumors already 2 h after injection was 37±19, which was significantly (p<0.001, Mann-Whitney test) higher than the value for HER2-negative tumors (5±2) (FIG. 3). There was a tendency of increase in the ratio with time (FIG. 3), but the difference between the time points was not significant. The tumor-to-contralateral breast ratio for 500 μg was significantly (p<0.05) higher than for 1000 μg at 2, 4 and 6 h. Moreover, the difference in tumor-to-contralateral breast ratio value between HER2-positive and HER2-negative tumors in the case of injection of 1000 μg was not significant (p>0.05, Mann-Whitney test) at any time point (FIG. 3). The tumor-to-contralateral breast ratio for 250 μg at 2 h (7.8±4.9) was also significantly (p<0.05) lower than for 500 μg (FIG. 3).

Patient 17 was enrolled in this study because the initial IHC evaluation of the analyzed biopsy suggested a 3+ expression level. However, the image showed unusually low tumor-to-contralateral breast ratio (1.33 at 2 h). The biopsy samples were further evaluated and were found FISH-negative. As a consequence, the treatment was adjusted and the HER2-targeting therapy was cancelled.

Imaging of patient 4 revealed, besides a primary tumor and auxiliary metastases, a site of accumulation in rib 5 and two sites at vertebra Th 8 and Th9 (FIG. 5A). CT imaging and bone scan using ^{99m}Tc-pyrophosphate (FIG. 5B) did not reveal any metastases. However, further evaluation with MRI confirmed the presence of metastases in Th 8 and Th9. As a consequence of these findings, the treatment strategy of Patient 4 was changed to initiate chemotherapy and HER2-targeting therapy instead of surgical treatment. A bone scan using ^{99m}Tc-pyrophosphate (FIG. 5C) confirmed the presence of metastatic lesions in rib 5 and Th8 and Th9 vertebra six months after imaging using ^{99m}Tc-ADAPT6.

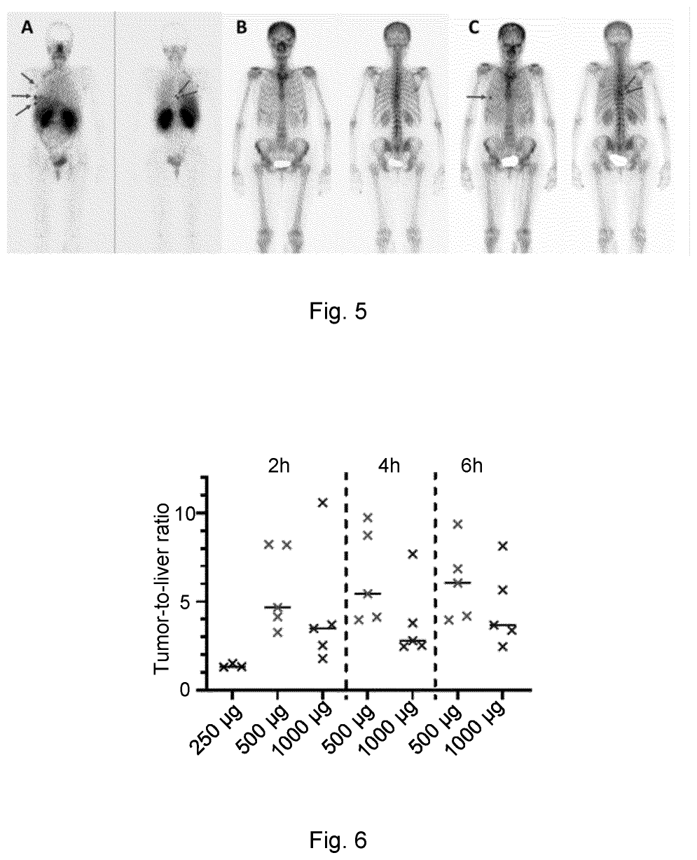

Injections of ^{99m}Tc-ADAPT6 resulted in higher uptake in tumors than in liver, regardless of the injected dose (FIG. 6). A slightly higher tumor-to-liver ratios for the injected protein dose of 500 μg could be seen.

### DISCUSSION

The results of the present study demonstrate that injections of ^{99m}Tc-ADAPT6 are safe and well tolerated. The mean effective dose of 0.010 mSv/MBq in this study corresponds to 3.8 mSv per patient. This is slightly lower compared to doses reported from imaging using ⁶⁸Ga-ABY25 affibody molecule (5.6 mSv) (Sandström 2016) or ⁶⁸Ga-nanobody (4.6 mSv) (Keyaerts 2016), and appreciably lower than effective doses for ⁸⁹Zr-trastuzumab (18-38 mSv) (Dijkers 2010; Laforest 2016) or ⁸⁹Zr-pertuzumab (39 mSv) (Ulaner 2018). Noteworthy is that clear discrimination between HER2-positive and HER2-negative tumors already 2 h after injection might permit further two-fold reduction of injection activity.

Discrimination between HER2-positive and HER2-negative lesions is the ultimate goal of molecular imaging. However, the term “HER2-negative”, i.e. unsuitable for treatment with HER2-targeting therapies, is deceptive. Breast tumors with IHC score of 2+ (and FISH negative) are considered as HER2-negative, but they may express up to 500 000 HER2 receptors per cell (Ross 2004). Thus, some accumulation of imaging probes is expected even in HER2-negative lesions. Studies in mice have demonstrated that an increase of the injected dose of ⁶⁸Ga-labeled ADAPT6 from 1 to 15 μg improved discrimination between human xenografts with high and low HER2 expression, although at the cost of slightly lower uptake in tumors with high expression (Garousi 2015). However, the translation from mice to humans is quite unpredictable. Therefore, injections of ^{99m}Tc-ADAPT6 at different dose levels were evaluated. Surprisingly, 500 μg provided excellent discrimination already 2 h after injection (FIG. 3), and the tumor-to-contralateral ratio had a tendency to increase over time. On the contrary, injection of 1000 μg did not enable discrimination between HER2 positive and negative tumors. To check if a further reduced amount of injected amount would improve the discrimination, an additional smaller cohort of patients was injected with 250 μg ^{99m}Tc-ADAPT6. However, the contrast of such imaging was clearly inferior to the contrast of imaging using 500 μg ^{99m}Tc-ADAPT6 (FIG. 3). Thus, the injected dose of 500 μg is optimal, and deviation from this dose would result in a decrease of both sensitivity and specificity of imaging of HER2-expression. The 500 μg dose appears to strike a balance between the saturation of HER2 in liver, which increases the bioavailability of radiolabeled ADAPT6, and the saturation of HER2 in tumors, which decreases ^{99m}Tc-ADAPT6 uptake in HER2-positive lesions. The capacity of ^{99m}Tc-ADAPT6 to make a clear discrimination already at 2 h after injection is unusual. For example, ⁶⁸Ga-labeled affibody molecule provides such discrimination after 4 h (Sörensen 2016). The capacity of early imaging enables the reduction of injected activity and, accordingly, a lower effective dose to patients. Obviously, clinical imaging using ^{99m}Tc-ADAPT6 is preferably performed around 2 h after injection. Increasing the time interval between injection and imaging may require either increasing the injected activity (and, consequently, the effective dose) or decreasing counting statistics at the time of injection (and, therefore, decrease of reconstruction fidelity).

PET is considered to be the imaging modality that provides the best resolution and sensitivity. However, the modern PET/CT facilities are mainly installed in Europe and North America, while SPECT is the most common imaging modality in Asia and South America. Therefore, there is a need for ^{99m}Tc-labeled targeting proteins and peptides in these regions (Briganti 2019). Moreover, the development of CZT-based cameras improves SPECT imaging in terms of resolution and sensitivity appreciably (Desmonts 2020; Goshen 2018). Hence, an increased use of CZT SPECT for molecular imaging even in Europe and US can be foreseen. The imaging method of the present disclosure is a viable option for such applications.

For the reasons set out below, it is expected that a dose of around 500 μg will be optimal also in case ^{99m}Tc is replaced with another radionuclide.

The major factors determining the tumor uptake are: injected protein dose; extravasation rate in tumors; diffusion rate in tumors; clearance rate of imaging agent that is not bound to a tumor or HER2 in normal tissue; binding to HER2 in tumor; and binding to HER2 expressed in normal hepatocytes in liver. Extravasation, diffusion and clearance rates are determined mainly by the size of the agent. The type of radiolabel does not affect the size to any significant extent. The binding to HER2 (in tumor or hepatocytes) is determined by affinity, which is primarily determined by the HER2-binding protein. In is not expected that the type of radiolabel has any major impact on the affinity, in particular when the radionuclide is separated from the HER2-binding region by a spacer region.

### REFERENCES

Bartley A N, Washington M K, Colasacco C, et al. HER2 Testing and Clinical Decision Making in Gastroesophageal Adenocarcinoma: Guideline From the College of American Pathologists, American Society for Clinical Pathology, and the American Society of Clinical Oncology. J Clin Oncol. 2017 February; 35(4):446-464
Bensch F, Brouwers A H, Lub-de Hooge M N, de Jong J R, van der Vegt B, Sleijfer S, et al. 89Zr-trastuzumab PET supports clinical decision making in breast cancer patients, when HER2 status cannot be determined by standard work up. Eur J Nucl Med Mol Imaging. 2018; 45:2300-6.
Briganti V, Cuccurullo V, Di Stasio G D, Mansi L. Gamma Emitters in Pancreatic Endocrine Tumors Imaging in the PET Era: Is there a Clinical Space for ^{99m}Tc-peptides? Curr Radiopharm. 2019; 12(2):156-170
Desmonts C, Bouthiba M A, Enilorac B, Nganoa C, Agostini D, Aide N. Evaluation of a new multipurpose whole-body CzT-based camera: comparison with a dual-head Anger camera and first clinical images. EJNMMI Phys. 2020 Mar. 17; 7(1):18.
Dijkers E C, Oude Munnink T H, Kosterink J G, Brouwers A H, Jager P L, de Jong J R, et al. Biodistribution of 89Zr-trastuzumab and PET imaging of HER2-positive lesions in patients with metastatic breast cancer. Clin Pharmacol Ther. 2010; 87:586-92.
Garousi J, Lindbo S, Nilvebrant J, et al. ADAPT, a Novel Scaffold Protein-Based Probe for Radionuclide Imaging of Molecular Targets That Are Expressed in Disseminated Cancers. Cancer Res. 2015; 75:4364-4371
Gebhart G, Flamen P, De Vries E G, Jhaveri K, Wimana Z. Imaging Diagnostic and Therapeutic Targets: Human Epidermal Growth Factor Receptor 2. J Nucl Med. 2016 February; 57 Suppl 1:81S-8S
Gebhart G, Lamberts L E, Wimana Z, Garcia C, Emonts P, Ameye L, et al. Molecular imaging as a tool to investigate heterogeneity of advanced HER2-positive breast cancer and to predict patient outcome under trastuzumab emtansine (T-DM1): the ZEPHIR trial. Ann Oncol 2016; 27:619-24.
Goshen E, Beilin L, Stern E, Kenig T, Goldkorn R, Ben-Haim S. Feasibility study of a novel general purpose CZT-based digital SPECT camera: initial clinical results. EJNMMI Phys. 2018 Mar. 14; 5(1):6
Keyaerts M, Xavier C, Heemskerk J, Devoogdt N, Everaert H, Ackaert C, Vanhoeij M, Duhoux F P, Gevaert T, Simon P, Schallier D, Fontaine C, Vaneycken I, Vanhove C, De Greve J, Lamote J, Caveliers V, Lahoutte T. Phase I Study of 68Ga-HER2-Nanobody for PET/C T Assessment of HER2 Expression in Breast Carcinoma. J Nucl Med. 2016 January; 57(1):27-33
Laforest R, Lapi S E, Oyama R, Bose R, Tabchy A, Marquez-Nostra B V, et al. [89Zr]Trastuzumab: Evaluation of Radiation Dosimetry, Safety, and Optimal Imaging Parameters in Women with HER2-Positive Breast Cancer. Mol Imaging Biol. 2016; 18:952-9
Lindbo S, Garousi J, Åstrand M, Honarvar H, Orlova A, Hober S, Tolmachev V. Influence of Histidine-Containing Tags on the Biodistribution of ADAPT Scaffold Proteins. Bioconjug Chem. 2016 Mar. 16; 27(3):716-26.
Mankoff D A, Edmonds C E, Farwell M D, Pryma D A. Development of Companion Diagnostics. Semin Nucl Med 2016; 46:47-56.
Mortimer J E, Bading J R, Colcher D M, Conti P S, Frankel P H, Carroll M I, et al. Functional imaging of human epidermal growth factor receptor 2-positive metastatic breast cancer using (64)Cu-DOTA-trastuzumab PET. J Nucl Med 2014; 55:23-9.
Nilvebrant J, Hober S. The albumin-binding domain as a scaffold for protein engineering. Comput Struct Biotechnol J. 2013; 6:1-8.
Nilvebrant J, Åstrand M, Georgieva-Kotseva M, Björnmalm M, Löfblom J, Hober S. Engineering of bispecific affinity proteins with high affinity for ERBB2 and adaptable binding to albumin. PLoS One. 2014 Aug. 4; 9(8):e103094.
Ross J S, Fletcher J A, Bloom K J, et al. Targeted therapy in breast cancer: the HER-2/neu gene and protein. Mol Cell Proteomics. 2004; 3:379-398.
Sandström M, Lindskog K, Velikyan I, Wennborg A, Feldwisch J, Sandberg D, Tolmachev V, Orlova A, Sörensen J, Carlsson J, Lindman H, Lubberink M. Biodistribution and Radiation Dosimetry of the Anti-HER2 Affibody Molecule 68Ga-ABY-025 in Breast Cancer Patients. J Nucl Med. 2016 June; 57(6):867-71.
Sörensen J, Velikyan I, Sandberg D, Wennborg A, Feldwisch J, Tolmachev V, Orlova A, Sandström M, Lubberink M, Olofsson H, Carlsson J, Lindman H. Measuring HER2-Receptor Expression In Metastatic Breast Cancer Using [68Ga]ABY-025 Affibody PET/CT. Theranostics. 2016 Jan. 1; 6(2):262-71.
Tolmachev V. Imaging of HER-2 overexpression in tumors for guiding therapy. Curr Pharm Des 2008; 14:2999-3019.
Ulaner G A, Hyman D M, Lyashchenko S K, Lewis J S, Carrasquillo J A. 89Zr-Trastuzumab PET/CT for Detection of Human Epidermal Growth Factor Receptor 2-Positive Metastases in Patients With Human Epidermal Growth Factor Receptor 2-Negative Primary Breast Cancer. Clin Nucl Med. 2017; 42:912-7.
Ulaner G A, Lyashchenko S K, Riedl C, Ruan S, Zanzonico P B, Lake D, et al. First-in-Human Human Epidermal Growth Factor Receptor 2-Targeted Imaging Using 89Zr-Pertuzumab PET/C T: Dosimetry and Clinical Application in Patients with Breast Cancer. J Nucl Med. 2018; 59:900-6.
Wolff A C, Hammond M E, Hicks D G, et al. Recommendations for human epidermal growth factor receptor 2 testing in breast cancer: American Society of Clinical Oncology/College of American Pathologists clinical practice guideline update. J Clin Oncol. 2013; 31:3997-4013.

1-19. (canceled)

20. A unit dose comprising an imaging agent in an amount of 400-700 μg, wherein the imaging agent is a conjugate comprising a radionuclide and a HER2-binding protein (HBP) and wherein the HBP comprises an amino acid sequence selected from
i) LAX₃AKX₆TX₈X₉Y HLX₁₃X₁₄X₁₅GVX₁₈DX₂₀ YKX₂₃LIDKX₂₈KT VEX₃₃VX₃₅AX₃₇YX₃₉X₄₀ ILX₄₃ALP, wherein, independently of each other,
X₃ is selected from A, G, P, S and V;
X₆ is selected from D and E;
X₈ is selected from A and V;
X₉ is selected from L and N;
X₁₃ is selected from D and T;
X₁₄ is selected from K and R;
X₁₅ is selected from I, L, M, T and V;
X₁₈ is selected from S and A;
X₂₀ is selected from F, Y and A;
X₂₃ is selected from D and R;
X₂₈ is selected from A and V;
X₃₃ is selected from G, S and D;
X₃₅ is selected from K, M and R;
X₃₇ is selected from L and R;
X₃₉ is selected from A, F and L;
X₄₀ is selected from A and E; and
X₄₃ is selected from A, H, K, P, R, T, Q and Y;
and ii) an amino acid sequence which has at least 95% identity to the sequence defined in i).

21. The unit dose of claim 20, wherein the imaging agent is formulated in a composition adapted for intravenous administration.

22. The unit dose of claim 21, wherein the volume of the composition is 1-15 ml.

23-24. (canceled)

25. The unit dose of claim 20, wherein the amount is 400-600 μg.

26. A product comprising a container and the unit dose of claim 20, wherein the unit dose is contained in the container.

27. The product of claim 26, wherein the container is a vial or ampoule.

28. (canceled)

29. A method of visualization of HER2 expression in a human patient, said method comprising an administration of an imaging agent to the patient in a dose of 400-700 μg and subsequently a scanning of the patient to visualize HER2 expression, wherein the imaging agent is a conjugate comprising a radionuclide and a HER2-binding protein (HBP) and wherein the HBP comprises or consists of an amino acid sequence selected from
i) LAX₃AKX₆TX₈X₉Y HLX₁₃X₁₄X₁₅GVX₁₈DX₂₀ YKX₂₃LIDKX₂₈KT VEX₃₃VX₃₅AX₃₇YX₃₉X₄₀ ILX₄₃ALP, wherein, independently of each other,
X₃ is selected from A, G, P, S and V;
X₆ is selected from D and E;
X₈ is selected from A and V;
X₉ is selected from L and N;
X₁₃ is selected from D and T;
X₁₄ is selected from K and R;
X₁₅ is selected from I, L, M, T and V;
X₁₈ is selected from S and A;
X₂₀ is selected from F, Y and A;
X₂₃ is selected from D and R;
X₂₈ is selected from A and V;
X₃₃ is selected from G, S and D;
X₃₅ is selected from K, M and R;
X₃₇ is selected from L and R;
X₃₉ is selected from A, F and L;
X₄₀ is selected from A and E; and
X₄₃ is selected from A, H, K, P, R, T, Q and Y;
and ii) an amino acid sequence which has at least 95% identity to the sequence defined in i).

30. The method of claim 29, wherein the radionuclide is coupled to the N-terminal end of the HBP.

31. The method of claim 30, wherein the imaging agent further comprises a linking amino acid sequence and the radionuclide is coupled to the N-terminal end of the HBP via the linking amino acid sequence.

32. (canceled)

33. The method of claim 31, wherein at least part of the linking amino acid sequence forms a chelator for the radionuclide.

34. (canceled)

35. The method of claim 31, wherein the linking amino acid sequence distances any chelator or other radionuclide-binding moiety from the HBP by at least five amino acid residues, such as at least six amino acid residues.

36. The method of claim 29, wherein, in amino acid sequence i):
X₃ is selected from A, G, P;
X₆ is E;
X₉ is L;
X₁₃ is D;
X₁₄ is R;
X₁₅ is selected from L and V;
X₁₈ is selected from S and A;
X₂₀ is selected from F, Y and A;
X₂₈ is A;
X₃₃ is G;
X₃₅ is selected from K and R;
X₃₇ is L;
X₃₉ is selected from F and L;
X₄₀ is E; and
X₄₃ is selected from H, P and R.

37. The method of claim 29, wherein the HBP comprises or consists of an amino acid sequence selected from the group consisting of:
(SEQ ID NO: 6)
LAAAKETALY HLDRLGVADA YKDLIDKAKT VEGVKARYFE ILHALP;
(SEQ ID NO: 7)
LAAAKETALY HLDRVGVSDY YKDLIDKAKT VEGVRALYLE ILPALP;
(SEQ ID NO: 8)
LAPAKETALY HLDRVGVSDY YKDLIDKAK TVEGVRALYFE ILHALP;
(SEQ ID NO: 9)
LAAAKETALY HLDRLGVSDY YKDLIDKAK TVEGVKALYFE ILHALP;
(SEQ ID NO: 10)
LAPAKETALY HLDRLGVSDY YKDLIDKAK TVEGVRALYLE ILKALP;
(SEQ ID NO: 11)
LAGAKETALY HLDRLGVSDY YKDLIDKAK TVEGVRALYLE ILTALP;
(SEQ ID NO: 12)
LAPAKETALY HLDRLGVSDY YKDLIDKAK TVEGVRALYFE ILRALP;
(SEQ ID NO: 13)
LAGAKETALY HLDRVGVSDY YKDLIDKAK TVEGVRALYLE ILRALP;
(SEQ ID NO: 14)
LAAAKETALY HLDRVGVSDY YKDLIDKAK TVEGVMALYAE ILPALP;
(SEQ ID NO: 15)
LAGAKETALY HLDKTGVSDY YKDLIDKAK TVEGVRALYLE ILQALP;
(SEQ ID NO: 16)
LAAAKETALY HLTRVGVSDY YKDLIDKAK TVEGVRALYFE ILYALP;
and
(SEQ ID NO: 17)
LASAKDTALY HLDRVGVSDY YKDLIDKAK TVEGVRALYAE ILAALP.

38-41. (canceled)

42. The method of claim 29, wherein the imaging agent comprises less than 73 amino acid residues.

43. The method of claim 29, wherein the administration is intravenous.

44. The method of claim 29, wherein the scanning is carried out within 4 hours of the administration of the imaging agent.

45. The method of claim 44, wherein the scanning is carried out between 1 and 3 hours after the administration of the imaging agent.

46-47. (canceled)

48. The unit dose of claim 20, wherein the amount is 450-550 μg.

49. The unit dose of claim 20, wherein the amount is about 500 μg.

50. The method of claim 29, wherein the imaging agent comprises less than 68 amino acid residues.


SEQUENCE LISTING (18 sequences)

SEQ ID NO: 1 - protein, 63 aa, Homo sapiens
GSSHHHHHHDEAVDANSLAAAKETALYHLDRLGVADAYKDLIDKAKTVEGVKARYFEILHALP

SEQ ID NO: 2 - protein, 17 aa, Homo sapiens
GSSHHHHHHDEAVDANS

SEQ ID NO: 3 - protein, 6 aa, Homo sapiens
HHHHHH

SEQ ID NO: 4 - protein, 8 aa, Homo sapiens
DEAVDANS

SEQ ID NO: 5 - protein, 6 aa, Homo sapiens
HEHEHE

SEQ ID NO: 6 - protein, 46 aa, Homo sapiens
LAAAKETALYHLDRLGVADAYKDLIDKAKTVEGVKARYFEILHALP

SEQ ID NO: 7 - protein, 46 aa, Homo sapiens
LAAAKETALYHLDRVGVSDYYKDLIDKAKTVEGVRALYLEILPALP

SEQ ID NO: 8 - protein, 46 aa, Homo sapiens
LAPAKETALYHLDRVGVSDYYKDLIDKAKTVEGVRALYFEILHALP

SEQ ID NO: 9 - protein, 46 aa, Homo sapiens
LAAAKETALYHLDRLGVSDYYKDLIDKAKTVEGVKALYFEILHALP

SEQ ID NO: 10 - protein, 46 aa, Homo sapiens
LAPAKETALYHLDRLGVSDYYKDLIDKAKTVEGVRALYLEILKALP

SEQ ID NO: 11 - protein, 46 aa, Homo sapiens
LAGAKETALYHLDRLGVSDYYKDLIDKAKTVEGVRALYLEILTALP

SEQ ID NO: 12 - protein, 46 aa, Homo sapiens
LAPAKETALYHLDRLGVSDYYKDLIDKAKTVEGVRALYFEILRALP

SEQ ID NO: 13 - protein, 46 aa, Homo sapiens
LAGAKETALYHLDRVGVSDYYKDLIDKAKTVEGVRALYLEILRALP

SEQ ID NO: 14 - protein, 46 aa, Homo sapiens
LAAAKETALYHLDRVGVSDYYKDLIDKAKTVEGVMALYAEILPALP

SEQ ID NO: 15 - protein, 46 aa, Homo sapiens
LAGAKETALYHLDKTGVSDYYKDLIDKAKTVEGVRALYLEILQALP

SEQ ID NO: 16 - protein, 46 aa, Homo sapiens
LAAAKETALYHLTRVGVSDYYKDLIDKAKTVEGVRALYFEILYALP

SEQ ID NO: 17 - protein, 46 aa, Homo sapiens
LASAKDTALYHLDRVGVSDYYKDLIDKAKTVEGVRALYAEILAALP

SEQ ID NO: 18 - protein, 46 aa, Homo sapiens
  SITE (3)..(3): Xaa is selected from A, G, P, S and V
  SITE (6)..(6): Xaa is selected from D and E
  SITE (8)..(8): Xaa is selected from A and V
  SITE (9)..(9): Xaa is selected from L and N
  SITE (13)..(13): Xaa is selected from D and T
  SITE (14)..(14): Xaa is selected from K and R
  SITE (15)..(15): Xaa is selected from I, L, M, T and V
  SITE (18)..(18): Xaa is selected from S and A
  SITE (20)..(20): Xaa is selected from F, Y and A
  SITE (23)..(23): Xaa is selected from D and R
  SITE (28)..(28): Xaa is selected from A and V
  SITE (33)..(33): Xaa is selected from G, S and D
  SITE (35)..(35): Xaa is selected from K, M and R
  SITE (37)..(37): Xaa is selected from L and R
  SITE (39)..(39): Xaa is selected from A, F and L
  SITE (40)..(40): Xaa is selected from A and E
  SITE (43)..(43): Xaa is selected from A, H, K, P, R, T, Q and Y
LAXAKXTXXYHLXXXGVXDXYKXLIDKXKTVEXVXAXYXXILXALP